(19) United States
(12) Patent Application Publication
(10) Pub. No.: US 2025/0204868 A1
(43) Pub. Date: Jun. 26, 2025

(54) DISPLAY APPARATUS AND METHOD OF OPERATING THE SAME

(71) Applicant: SAMSUNG DISPLAY CO., LTD., YONGIN-SI (KR)

(72) Inventor: CHUL KIM, Yongin-si (KR)

(21) Appl. No.: 18/977,412

(22) Filed: Dec. 11, 2024

(30) Foreign Application Priority Data
Dec. 20, 2023 (KR) 10-2023-0187719

(51) Int. Cl. A61B 5/00; A61B 5/024; G09G 3/3233

(52) U.S. Cl. CPC A61B 5/7445; A61B 5/02427; G09G 3/3233; G09G 2300/0819; G09G 2300/0842; G09G 2300/0861; G09G 2354/00

(57) ABSTRACT

A display apparatus includes a display panel that includes a plurality of pixels and a panel driver that drives the display panel. The pixels include an adjacent horizontal pixel group that includes first to second pixel-rows and an adjacent vertical pixel group that includes first to second pixel-columns. The pixels include an emitting pixel that includes a light emitting element and a light sensing pixel that includes a photodiode. The panel driver drives the display panel to display a first guidance image that guides a user to place fingers on a first sensing region and a second sensing region of the display panel. The light sensing pixel outputs a sensing current that generates photoplethysmography (PPG) signals by sensing the fingers of the user. The first sensing region and the second sensing region are displayed on one of the adjacent horizontal pixel group or the adjacent vertical pixel group.

### CROSS-REFERENCE TO RELATED APPLICATION(S)

This application claims priority under 35 USC §119 from Korean Patent Application No. 10-2023-0187719, filed on Dec. 20, 2023 in the Korean Intellectual Property Office (KIPO), the contents of which are herein incorporated by reference in their entirety.

### TECHNICAL FIELD

Embodiments of the present inventive concept are directed to a display device. More particularly, embodiments of the present inventive concept are directed to a display apparatus that provides a biomarker and a method of operating the display apparatus.

### DISCUSSION OF THE RELATED ART

An electronic device, such as a smart phone, a smart watch, etc., that performs a bio-sensing operation, such as a fingerprint sensing operation, a photoplethysmography (PPG) sensing operation, etc., is being developed. In general, an electronic device can perform a bio-sensing operation by using a sensor separate from a display device. In this case, a size of a display region of the electronic device or the display device may be reduced, and a size of a bezel may be increased.

To address this situation, an in-cell light sensor technique that employs an optical sensor or a light sensing pixel within the display region of the display device has been recently developed.

### SUMMARY

Some embodiments provide a method of providing a biomarker by a display apparatus.

Some embodiments provide a display apparatus that provides a biomarker.

According to embodiments, a display apparatus includes a display panel that includes a plurality of pixels and a panel driver that drives the display panel. The pixels include an adjacent horizontal pixel group that includes first to second pixel-rows and an adjacent vertical pixel group that includes first to second pixel-columns. The pixels include an emitting pixel that includes a light emitting element and a light sensing pixel that includes a photodiode. The panel driver drives the display panel such that the display panel displays a first guidance image that guides a user to place fingers on a first sensing region and a second sensing region of the display panel. The light sensing pixel outputs a sensing current that generates photoplethysmography (PPG) signals by sensing the user's fingers. The first sensing region and the second sensing region are displayed on one of the adjacent horizontal pixel group or the adjacent vertical pixel group.

In an embodiment, the first sensing region and the second sensing region are displayed on the adjacent vertical pixel group.

In an embodiment, the panel driver includes a gate driver that applies a gate signal to the display panel through a plurality of gate lines and a light sensing driver that receives the sensing current from the pixels through a plurality of sensing lines. The gate lines include an adjacent gate line group that includes first to second gate lines. The sensing lines include an adjacent sensing line group that includes first to second sensing lines. The adjacent vertical pixel group is connected to the adjacent sensing line group.

In an embodiment, the light sensing driver receives the sensing current from the adjacent sensing line group connected to the adjacent vertical pixel group.

In an embodiment, the sensing lines include first to P-th sensing lines, where P is a positive integer. The first to P-th sensing lines are sequentially disposed on the display panel. The adjacent sensing line group includes at least two adjacent sensing lines of the first to P-th sensing lines.

In an embodiment, the gate lines extend in a first direction and the sensing lines extend in a second direction different from the first direction.

In an embodiment, the panel driver drives the display panel such that the display panel displays a second guidance image that guides the user to place fingers on the first sensing region, the second sensing region, a third sensing region and a fourth sensing region of the display panel. The third sensing region and the fourth sensing region are displayed on the adjacent vertical pixel group.

In an embodiment, the first sensing region and the second sensing region are displayed on the adjacent horizontal pixel group.

In an embodiment, the panel driver includes a gate driver that applies a gate signal to the display panel through a plurality of gate lines and a light sensing driver that receives the sensing current from the pixels through a plurality of sensing lines. The gate lines include an adjacent gate line group. The sensing lines include an adjacent sensing line group. The adjacent horizontal pixel group is connected to the adjacent gate line group.

In an embodiment, the light sensing driver receives the sensing current from the sensing lines connected to the adjacent horizontal pixel group.

In an embodiment, the sensing lines include first to N-th gate lines, where N is a positive integer. The first to N-th gate lines are sequentially disposed on the display panel. The adjacent gate line group includes at least two adjacent gate lines of the first to N-th gate lines.

In an embodiment, the gate lines extend in a first direction and the sensing lines extend in a second direction different from the first direction.

In an embodiment, the panel driver drives the display panel such that the display panel displays a third guidance image that guides the user to place fingers on the first sensing region, the second sensing region, a third sensing region and a fourth sensing region of the display panel. The third sensing region and the fourth sensing region are displayed on the adjacent horizontal pixel group.

In an embodiment, the panel driver drives the display panel in a horizontal mode or a vertical mode. When the display panel displays the guidance image in the horizontal mode, the panel driver converts the display panel from the horizontal mode to the vertical mode in response to receipt of an app-on signal.

According to embodiments, a method of operating a display apparatus includes displaying a guidance image that guides a user to respectively place a first finger and a second finger on a first sensing region and a second sensing region that are displayed on one of an adjacent horizontal pixel group that includes first to second pixel-rows or an adjacent vertical pixel group that includes first to second pixel-columns, generating photoplethysmography (PPG) signals by performing a PPG sensing operation when the first finger and the second finger are respectively located on the first sensing region and the second sensing region, and displaying a biomarker of the user that is determined based on the PPG signals.

In embodiments, the method further includes converting a mode of the display apparatus from a horizontal mode to a vertical mode in response to receipt of an app-on signal when the display apparatus is driven in the horizontal mode and displaying the first sensing region and the second sensing region on the adjacent horizontal pixel group in the vertical mode.

In embodiments, the first sensing region and the second sensing region are displayed on the adjacent vertical pixel group.

In embodiments, the adjacent vertical pixel group includes at least two adjacent pixel-columns of first to P-th pixel-columns, where is a positive integer.

In embodiments, the first sensing region and the second sensing region are displayed on the adjacent horizontal pixel group.

In embodiments, the adjacent horizontal pixel group includes at least two adjacent pixel-rows of first to N-th pixel-rows, where N is a positive integer.

As described above, in a display apparatus according to embodiments, the display apparatus can sense a user's fingers to generate a biomarker of the user based on a photoplethysmography (PPG) signal. To generate the PPG signal, a plurality of sensing regions are displayed. The sensing regions are displayed on one of an adjacent horizontal pixel group or an adjacent vertical pixel group. Accordingly, an accuracy of the PPG signals is increased. In addition, the display apparatus can sense the user's fingers through the plurality of sensing regions, which further increases an accuracy of the biomarker.

### BRIEF DESCRIPTION OF THE DRAWINGS

FIG. 1 is a block diagram of a display apparatus according to an embodiment of the present inventive concept.

FIG. 2 illustrates a photoplethysmography (PPG) sensing operation performed by a display apparatus of FIG. 1.

FIG. 3 illustrates a biomarker displayed on a display panel of FIG. 1.

FIG. 4 is a circuit diagram of an emitting pixel and a light sensing pixel in a pixel of FIG. 1.

FIG. 5 illustrates a guidance image output by a display apparatus of FIG. 1.

FIG. 6 is a block diagram of a display panel, a gate driver and a light sensing driver that output a guidance image of FIG. 5.

FIG. 7 illustrates a guidance image output by a display apparatus of FIG. 1.

FIG. 8 is a block diagram of a display panel, a gate driver and a light sensing driver that output a guidance image of FIG. 7.

FIG. 9 is a graph of a PPG signal generated by a guidance image of FIG. 7.

FIG. 10 illustrates a vertical mode and a horizontal mode of a display apparatus of FIG. 1.

FIG. 11 illustrates mode conversion of a display apparatus of FIG. 1.

FIG. 12 illustrates a guidance image output by a display apparatus of FIG. 1.

FIG. 13 illustrates a guidance image output by a display apparatus of FIG. 1.

FIG. 14 is a flowchart of a method of generating a biomarker.

FIG. 15 is a flowchart of a method of generating a biomarker.

FIG. 16 is a block diagram of an electronic apparatus according to an embodiment of the present inventive concept.

FIG. 17 illustrates an example in which an electronic apparatus of FIG. 16 is implemented as a smart phone.

### DETAILED DESCRIPTION

Hereinafter, embodiments of the present inventive concept will be described in detail with reference to the accompanying drawings.

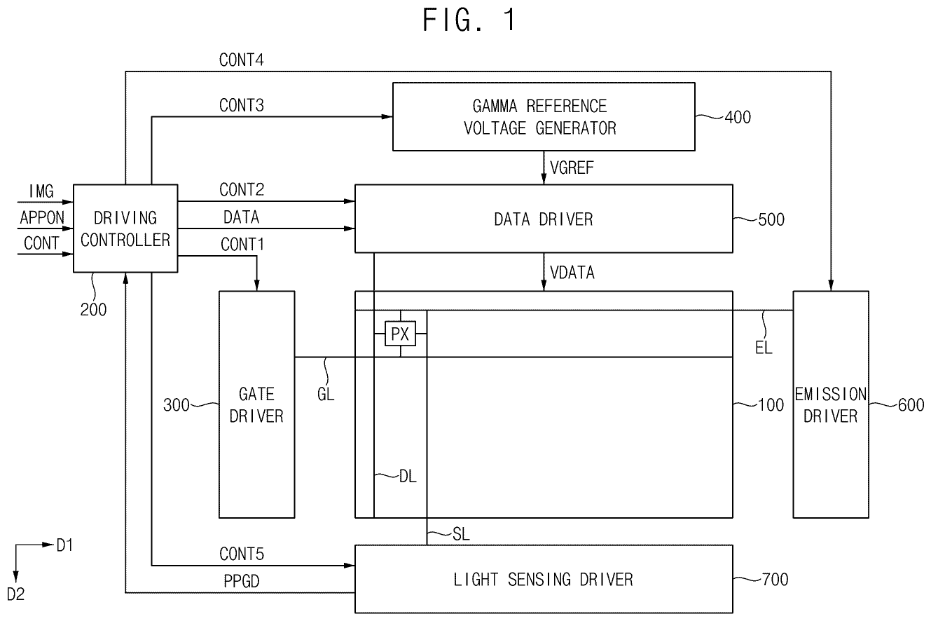

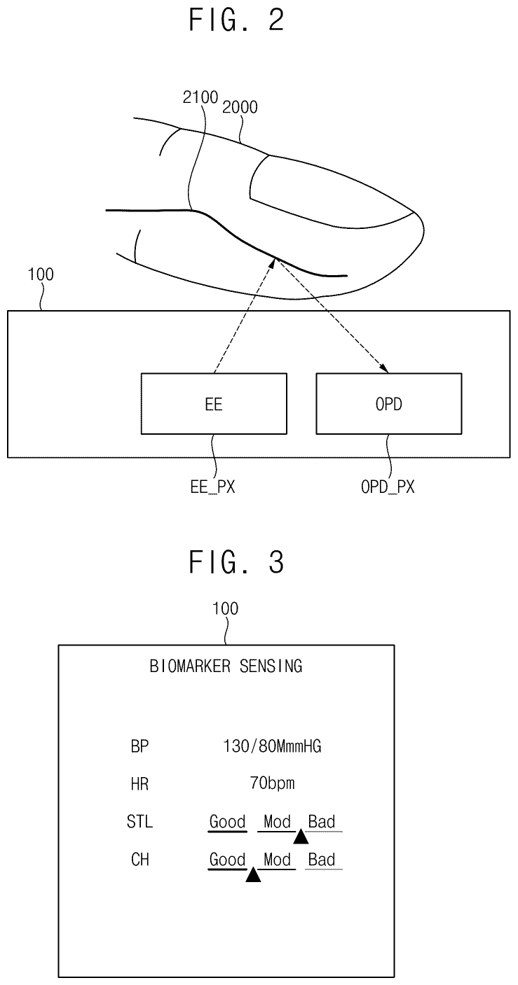

FIG. 1 is a block diagram of a display apparatus according to an embodiment of the present inventive concept. FIG. 2 illustrates an example of a photoplethysmography sensing operation performed by a display apparatus of FIG. 1. FIG. 3 illustrates an example of a biomarker displayed on a display panel of FIG. 1.

Referring to FIG. 1, in an embodiment, the display apparatus includes a display panel 100 that includes a plurality of pixels PX and a panel driver that drives the display panel 100. In an embodiment, the panel driver includes a gate driver 300 connected to the pixels PX through a plurality of gate lines GL, a data driver 500 connected to the pixels PX through a plurality of data lines DL, an emission driver 600 connected to the pixels PX through a plurality of emission lines EL, a light sensing driver 700 connected to the pixels PX through a plurality of sensing lines SL and a driving controller 200 that controls the gate driver 300, the data driver 500, the emission driver 600 and the light sensing driver 700.

The display panel 100 includes the gate lines GL, the data lines DL, the emission lines EL, the sensing lines SL and the pixels PX connected to the gate lines GL, the emission lines EL and the sensing lines SL. In some embodiments, the display panel 100 is an organic light emitting diode (OLED) display panel or a quantum dot (QD) display panel, but embodiments of the present inventive concept are not necessarily limited thereto.

In an embodiment, the gate lines GL extend in a first direction D1. The sensing line SL extends in a second direction D2 that crosses the first direction D1.

In an embodiment, the pixel PX includes a light emitting pixel EE_PX that includes a light emitting element EE and a light sensing pixel OPD_PX that includes an organic photodiode OPD.

The display apparatus includes the display panel 100, the driving controller 200, the gate driver 300, a gamma reference voltage generator 400, the data driver 500, the emission driver 600 and the light sensing driver 700. In an embodiment, the driving controller 200 and the data driver 500 are implemented as a single integrated circuit.

The display panel 100 includes a display region on which an image is displayed and a peripheral region adjacent to the display region. In an embodiment, the gate driver 300 is disposed in the peripheral region. In an embodiment, the gate driver 300 is integrated into the peripheral region.

The display panel 100 include the gate line GL, the data line DL, the emission line EL and the pixel PX electrically connected to the gate line GL, the data line DL and the emission line EL. The gate line GL and the data line DL extend in directions that cross each other.

The driving controller 200 receives input image data IMG, an app-on signal APPON and an input control signal CONT from a host processor, such as an application processor and/or a graphic processing unit, etc. In an embodiment, the input image data IMG includes red image data, green image data and blue image data. In an embodiment, the input image data IMG includes white image data. In an embodiment, the input image data IMG may include magenta image data, yellow image data and cyan image data. The input control signal CONT includes a master clock signal and a data enable signal. The input control signal CONT furthers include a vertical synchronizing signal and a horizontal synchronizing signal.

In an embodiment, the input image data IMG for sensing fingers of a user includes data of guidance image that guides the user to place fingers on a first sensing region and a second sensing region of display panel 100. The driving controller 200 outputs a data signal such that the guidance image is output in response to the app-on signal APPON.

The driving controller generates a first control signal CONT1, a second control signal CONT2, a third control signal CONT3, a fourth control signal CONT4, a fifth control signal CONT5 and a data signal DATA based on the input image data IMG and the input control signal CONT.

The driving controller 200 generates the first control signal CONT1 that controls an operation of the gate driver 300 based on the input control signal CONT, and outputs the first control signal CONT1 to the gate driver 300. The first control signal CONT1 includes a vertical start signal and a scan clock signal.

The driving controller 200 generates the third control signal CONT3 that controls an operation of the gamma reference voltage generator 400 based on the input control signal CONT, and outputs the third control signal CONT3 to the gamma reference voltage generator 400.

The driving controller 200 generates the second control signal CONT2 that controls an operation of the data driver 500 based on the input control signal CONT, and outputs the second control signal CONT2 to the data driver 500. The second control signal CONT2 includes a horizontal start signal and a load signal.

The driving controller 200 generates the data signal DATA based on the input image data IMG and the input control signal CONT. The driving controller 200 outputs the data signal DATA to the data driver 500.

The driving controller 200 generates the fourth control signal CONT4 that controls an operation of the emission driver 600 based on the input control signal CONT. The driving controller 200 outputs the fourth control signal CONT4 to the emission driver 600.

The driving controller 200 generates the fifth control signal CONT5 based on the input control signal CONT. The driving controller 200 outputs the fifth control signal CONT5 to the light sensing driver 700.

The gate driver 300 generates gate signals that drive the gate lines GL in response to the first control signal CONT1 received from the driving controller 200. The gate driver 300 outputs the gate signals to the gate lines GL.

The gamma reference voltage generator 400 generates a gamma reference voltage VGREF in response to the third control signal CONT3 received from the driving controller 200. The gamma reference voltage generator 400 provides the gamma reference voltage VGREF to the data driver 500. The gamma reference voltage VGREF has a value that corresponds to each of the data signals DATA. The gamma reference voltage generator 400 is disposed in the driving controller 200 or in the data driver 500.

The data driver 500 receives the second control signal CONT2 and the data signal DATA from the driving controller 200 and receives the gamma reference voltages VGREF from the gamma reference voltage generator 400. The data driver 500 converts the data signal DATA into analog data voltages VDATA using the gamma reference voltages VGREF. The data driver 500 outputs the data voltages VDATA to the data line DL.

In an embodiment, the data driver 500 is implemented with one or more integrated circuits. In an embodiment, the data driver 500 and the driving controller 200 are implemented as a single integrated circuit and the single integrated circuit may be called a timing controller embedded data driver (TED).

The emission driver 600 generates an emission signal that drives the emission line EL in response to the fourth control signal CONT4 received from the driving controller 200. The emission driver 600 outputs the emission signal to the emission line EL.

In an embodiment of the present inventive concept, the emission driver 600 is integrated onto the peripheral region of the display panel 100. In an embodiment of the present inventive concept, the emission driver 600 is mounted on the peripheral region of the display panel 100.

Although FIG. 1 shows that the gate driver 300 is disposed on a first side of the display panel 100 and the emission driver 600 is disposed on a second side of the display panel 100 for convenience of illustration, embodiments of the present inventive concept are not necessarily limited thereto. In some embodiments, the gate driver 300 and the emission driver 600 are disposed on the first side of the display panel 100. For example, the gate driver 300 and the emission driver 600 are disposed on the peripheral region of the display panel 100 on the same side of the display region of the display panel 100. For example, the gate driver 300 and the emission driver 600 are integrally formed with each other.

In an embodiment, the light sensing driver 700 receives the fifth control signal CONT5 from the driving controller 200. The light sensing driver 700 generates a sensing current by sensing the pixels PX through the sensing lines SL. In an embodiment, the light sensing driver 700 is implemented with one or more integrated circuits. In an embodiment, the light sensing driver 700 is disposed in the data driver 500 or in the driving controller 200.

In an embodiment, the panel driver performs a photoplethysmography (PPG) sensing operation with the display panel 100. The PPG sensing operation includes having the emitting pixel EE_PX emits light, and the light sensing pixel OPD_PX senses light reflected from a blood vessel 2100 of as user's finger 2000. For example, when a user's heart contracts and a volume of the blood vessel 2100 increases, the amount of hemoglobin in the blood vessel 2100 increases, a light intensity absorbed by the hemoglobin increases, and the light sensing pixel OPD_PX measures a relatively low light intensity of reflected light. In contrast, when the heart of the user expands (or relaxes) and the volume of the blood vessel 2100 decreases, the amount of hemoglobin in the blood vessel 2100 decreases, the light intensity absorbed by the hemoglobin decreases, and the light sensing pixel OPD_PX measures a relatively high light intensity of the reflected light. The light sensing driver 700 generates a PPG signal PPGD that indicates the volume of the blood vessel 2100 based on the light intensity measured by the light sensing pixel OPD_PX. In an embodiment, the pixel PX outputs the sensing current to the light sensing driver 700 to generate the PPG signal PPGD.

In an embodiment, a biomarker is generated based on the PPG signal PPGD generated by the light sensing driver 700.

In an embodiment, the display panel 100 displays the guidance image that senses the user's fingers 2000. The PPG signal PPGD that corresponds to each of the fingers 2000 is generated, and an accuracy of the biomarker is increased.

The display panel 100 displays, as the biomarker, a blood pressure BP, a heart rate HR, a stress level STL and a cardiovascular health CH of the user. For example, the blood pressure BP is determined by detecting a feature of the PPG signal PPGD and by performing machine learning on the feature of the PPG signal PPGD. The heart rate HR is determined based on a period of the PPG signal PPGD. The stress level STL is determined based on a change of the period of the PPG signal PPGD. The cardiovascular health CH is determined based on a crest time of the PPG signal PPGD, or a difference in blood pressure between left and right fingers. In other embodiments, the display panel 100 further displays, as the biomarker, a respiratory rate, a blood vessel age (or a blood vessel elasticity), and an oxygen saturation. For example, the breathing rate can be determined based on a period of a low frequency component of the PPG signal PPGD, the blood vessel age can be determined based on a wave shape of the PPG signal PPGD, and the oxygen saturation can be determined by an intensity difference between green reflected light and red reflected light.

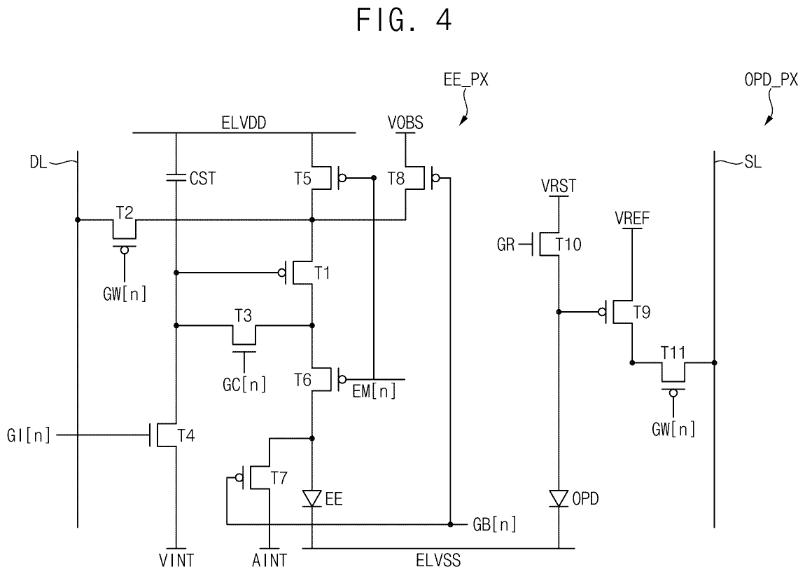

FIG. 4 is a circuit diagram of an example of an emitting pixel EE_PX and a light sensing pixel OPD_PX in a pixel PX of FIG. 1.

Referring to FIG. 4, in an embodiment, the emitting pixel EE_PX includes a first transistor T1 that generates a driving current, a second transistor T2 that outputs the data voltage VDATA of data line DL of FIG. 1 in response to a write signal GW[n], a third transistor T3 which diode-connects the first transistor T1 in response to a compensation signal GC[n], a fourth transistor T4 that outputs an initialization voltage VINT to a gate electrode of the first transistor T1 in response to a initialization signal GI[n], a fifth transistor T5 that connects a line of a first power voltage ELVDD and the first transistor T1 in response to the emission signal EM[n], a sixth transistor T6 that connects the first transistor T1 and the light emitting element EE in response to the emission signal EM[n], a seventh transistor T7 that outputs an anode initialization voltage AINT to the light emitting element EE in response to a bypass signal GB[n], an eighth transistor T8 that outputs a bias voltage VOBS to an electrode, such as the source, of the first transistor T1 in response to the bypass signal GB[n], a storage capacitor CST connected between the line of the first power voltage ELVDD and the gate electrode of the first transistor T1, and the light emitting element EE that emits light based on the driving current and has a cathode connected to a line of a second power voltage ELVSS.

According to embodiments, the light emitting element EE includes at least one of an organic light emitting diode (OLED), a nano light emitting diode (NED), a quantum dot (QD) light emitting diode, a micro light emitting diode, an inorganic light emitting diode, or any other suitable light emitting element. In some embodiments, as illustrated in FIG. 4, the first, second, fifth, sixth, seventh and eighth transistors T1, T2, T5, T6, T7 and T8 are P-type metal-oxide-semiconductor (PMOS) transistors, and the third and fourth transistors T3 and T4 are N-type metal-oxide-semiconductor (NMOS) transistors, but embodiments are not necessarily limited thereto.

Further, as illustrated in FIG. 4, the light sensing pixel OPD_PX includes a ninth transistor T9, a tenth transistor T10, an eleventh transistor T11 and the organic photodiode OPD.

The ninth transistor T9 generates a sensing current based on a voltage of an anode of the organic photodiode OPD. In some embodiments, the ninth transistor T9 includes a gate electrode connected to the anode of the organic photodiode OPD, a first electrode that receives a reference voltage VREF, and a second terminal.

The tenth transistor T10 resets the voltage of the anode of the organic photodiode OPD to a reset voltage VRST in response to a global reset signal GR. In some embodiments, the tenth transistor T10 includes a gate electrode that receives the global reset signal GR, a first electrode that receives the reset voltage VRST, and a second electrode connected to the anode of the organic photodiode OPD.

The eleventh transistor T11 outputs the sensing current generated by the ninth transistor T9 to the sensing line SL in response to the write signal GW[n]. In some embodiments, the eleventh transistor T11 includes a gate electrode that receives the write signal GW[n], a first electrode connected to the second electrode of the ninth transistor T9, and a second electrode connected to the sensing line SL.

The organic photodiode OPD measures a light intensity. For example, after the voltage of the anode of the organic photodiode OPD is reset to the reset voltage VRST, the voltage of the anode of the organic photodiode OPD can be increased by different amounts depending on the light intensity. The sensing current of the ninth transistor T9 is determined according to the voltage of the anode of the organic photodiode OPD, and the light sensing driver 700 generates a PPG signal that corresponds to the sensing current. In some embodiments, the organic photodiode OPD includes the anode connected to the gate electrode of the ninth transistor T9, and a cathode connected to the line of the second power voltage ELVSS.

Although FIG. 4 illustrates an embodiment of the emitting pixel EE_PX and the light sensing pixel OPD_PX, the emitting pixel EE_PX and the light sensing pixel OPD_PX of the display apparatus according to embodiments are not necessarily limited to the embodiment of FIG. 4, and have other structures in other embodiments.

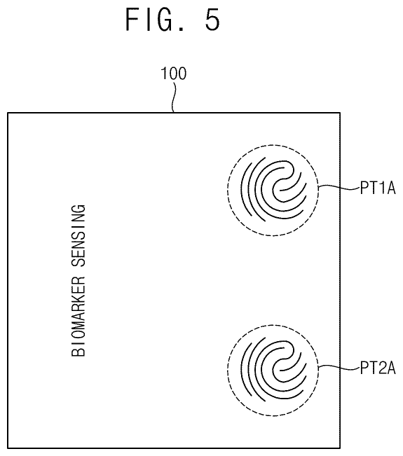

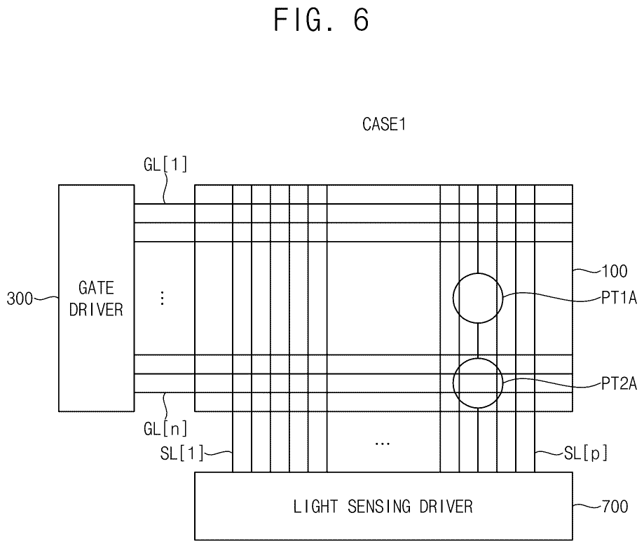

FIG. 5 illustrates an example of a guidance image CASE1 output by a display apparatus of FIG. 1. FIG. 6 is a block diagram of a display panel 100, a gate driver 300 and a light sensing driver 700 that output a guidance image CASE1 of FIG. 5.

Referring to FIGS. 1, 5 and 6, in an embodiment, the pixels PX include an adjacent vertical pixel group.

For example, an adjacent vertical pixel group can be defined as a plurality of adjacent pixel-columns. For example, the pixel-columns include first to P-th pixel columns, where Pis a positive integer. The adjacent vertical pixel group includes first to third pixel-columns. However, embodiments of the present inventive concept are not necessarily limited to this number of the pixel-columns in an adjacent vertical pixel group. For example, in some embodiments, the adjacent vertical pixel group includes the first to fifth pixel-columns. In addition, embodiments of the present inventive concept are not necessarily limited to a location of the adjacent vertical pixel group. For example, in some embodiments, the adjacent vertical pixel group includes the P-7-th to P-th pixel columns.

In an embodiment, the adjacent vertical pixel group is connected to an adjacent sensing line group. For example, the adjacent sensing line group may be defined as a plurality of adjacent sensing lines. The sensing lines include first to P-th sensing line SL[1] to SL[P]. For example, when the adjacent vertical pixel group includes first to third pixel-columns, the adjacent sensing line group includes first to third sensing lines. However, embodiments of the present inventive concept are not necessarily limited to the number of the pixel-columns included in the adjacent vertical pixel group and the location of the pixel-columns, so that embodiments of the present inventive concept are not necessarily limited to the number of sensing lines in the adjacent sensing line group or a location of the sensing lines in the adjacent sensing line group.

In an embodiment, some pixels in the adjacent vertical pixel group display a first sensing region PT1A and a second sensing region PT2A. For example, the first sensing region PT1A senses a user's first finger. For example, the second sensing region PT2A senses a user's different second finger.

In an embodiment, the first sensing region PT1A and the second sensing region PT2A are displayed on the adjacent sensing line group, so that the sensing lines SL located (or disposed) adjacently output the sensing current. Accordingly, an accuracy of the sensing current is increased, which increases an accuracy of the PPG signals PPGD. Accordingly, an accuracy of the biomarker can be increased.

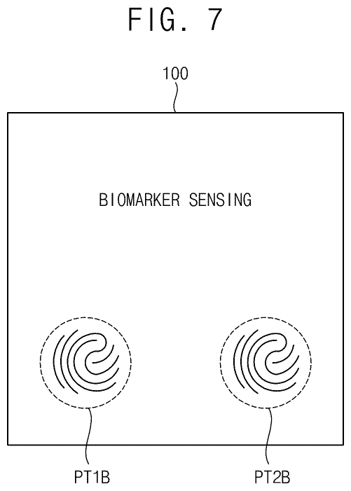

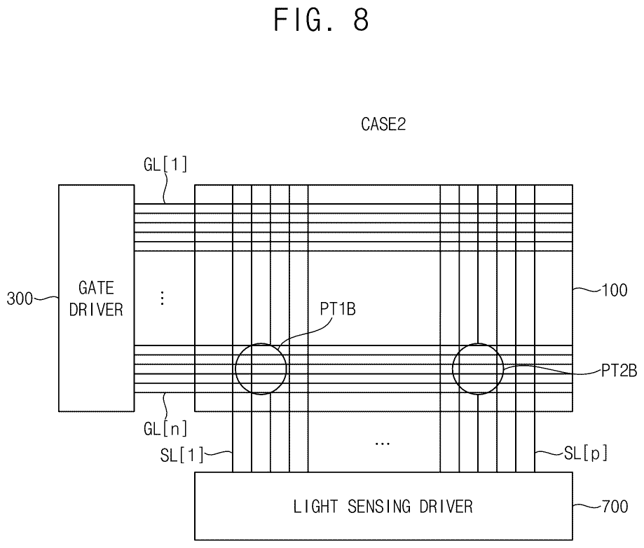

FIG. 7 illustrates an example of a guidance image CASE2 output by a display apparatus of FIG. 1. FIG. 8 is a block diagram of a display panel 100, a gate driver 300 and a light sensing driver 700 that output a guidance image CASE2 of FIG. 7.

Referring to FIGS. 1, 7 and 8, in an embodiment, the pixels PX includes an adjacent horizontal pixel group.

For example, an adjacent horizontal pixel group may be defined as a plurality of adjacent pixel-rows. For example, the pixel-rows include first to N-th pixel-rows, where Nis a positive integer. The adjacent horizontal pixel group includes first to third pixel-rows. However, embodiments of the present inventive concept are not necessarily limited to the number of the pixel-rows in the adjacent horizontal pixel group. For example, in some embodiments, the adjacent horizontal pixel group includes first to fifth pixel-rows. In addition, embodiments of the present inventive concept are not necessarily limited to a location of the adjacent horizontal pixel group. For example, in some embodiments, the adjacent horizontal pixel group includes the N-7-th to N-th pixel-rows.

In an embodiment, the adjacent horizontal pixel group is connected to an adjacent gate line group. For example, the adjacent gate line group may be defined as a plurality of adjacent gate lines. The sensing lines include first to N-th sensing line SL[1] to SL[n]. For example, when the adjacent horizontal pixel group includes first to third pixel-rows, the adjacent gate line group includes first to third gate lines. However, embodiments of the present inventive concept are not necessarily limited to the number of the pixel-rows in the adjacent horizontal pixel group or the location of the pixel-rows, so that embodiments of the present inventive concept are not necessarily limited to the number of the gate lines in the adjacent horizontal line group or a location of the gate lines in the adjacent horizontal line group.

In an embodiment, some pixels in the adjacent horizontal pixel group display a first sensing region PT1B and a second sensing region PT2B. For example, the first sensing region PT1B senses the user's first finger. For example, the second sensing region PT2B senses the user's different second finger.

In an embodiment, the first sensing region PT1B and the second sensing region PT2B are displayed on the adjacent gate line group. In an embodiment, the gate signal applied to the gate line is sequentially output to the first to N-th gate lines GL[1] to GL[n]. Accordingly, a timing of a gate signal applied to the first gate line GL[1] and a timing of a gate signal applied to the N-th gate line GL[n] differs.

In an embodiment, the first sensing region PT1B and the second sensing region PT2B are displayed on the adjacent gate line group, so that a timing of the gate signals applied to the first sensing region PT1B and the second sensing region PT2B is substantially same. Accordingly, an influence of deviations due to different gate signal timings can be reduced. Accordingly, an accuracy of the sensing current can be increased.

The accuracy of the sensing current can be increased, which increases an accuracy of the PPG signals PPGD. Accordingly, an accuracy of the biomarker may be increased.

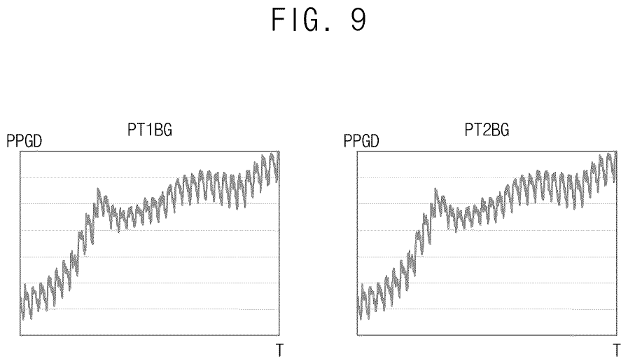

FIG. 9 is a graph that illustrates a PPG signal generated by a guidance image CASE2 of FIG. 7.

Referring to FIGS. 7 to 9, an X-axis of a first PPG signal graph PT1BG represents a time T and a Y-axis of the first PPG signal graph PT1BG represents the PPG signal PPGD. In addition, an X-axis of a second PPG signal graph PT2BG represents the time T and a Y-axis of the second PPG signal graph P21BG represents the PPG signal PPGD.

In an embodiment, the guidance image CASE2 displays the first sensing region PT1B and the second sensing region PT2B on some region of the adjacent horizontal pixel group. A first PPG signal that corresponds to the first sensing region PT1B is generated. A second PPG signal that corresponds to the second sensing region PT2B is generated. In addition, the PPG sensing operation is performed by those pixels PX located (or disposed) on the first sensing region PT1B and the second sensing region PT2B. Accordingly, an influence of different gate signal timings of the first PPG sensing signals that correspond to the first finger and the second PPG sensing signals that correspond to the second finger can be reduced. Accordingly, a result of the first PPG signal graph PT1BG and a result of the second PPG signal graph PT2BG are substantially the same.

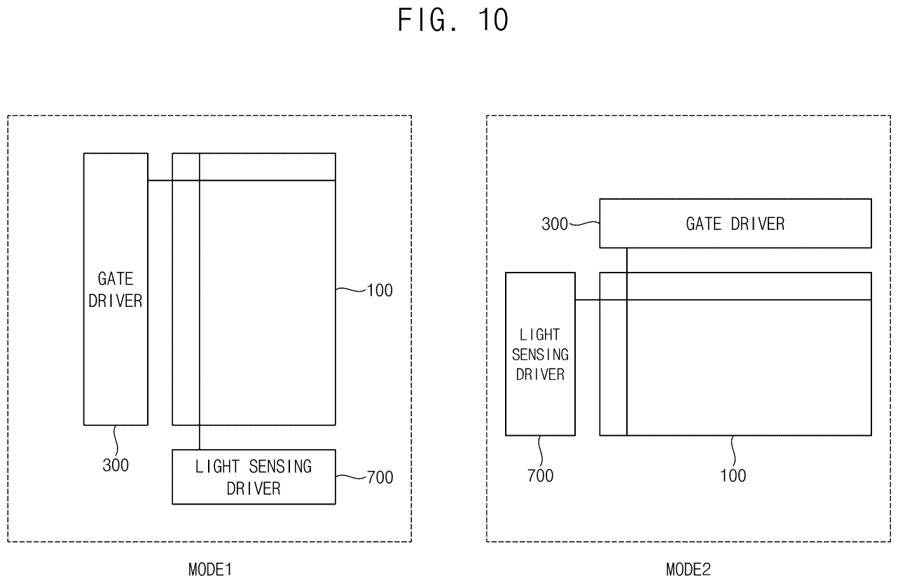

FIG. 10 illustrates a vertical mode MODE1 and a horizontal mode MODE2 of a display apparatus of FIG. 1.

Referring to FIGS. 1 and 10, in an embodiment, the panel driver drives the display panel 100 in a vertical mode MODE1 or a horizontal mode MODE2. For example, when the panel driver drives the display panel 100 in the vertical mode MODE1, the light sensing driver 700 is located at a first location or a second location of display panel 100. For example, the first location refers to either the top of the display panel 100 or the bottom of the display panel 100. For example, the second location refers to either the top of the display panel 100 or the bottom of the display panel 100. In addition, when the panel driver drives the display panel 100 in the vertical mode MODE1, the gate driver 300 is located at a third location of the display panel 100. For example, the third location is a side of the display panel 100.

For example, when the panel driver drives the display panel 100 in the horizontal mode MODE2, the light sensing driver 700 is located at the third location of the display panel 100. In addition, when the panel driver drives the display panel 100 in the horizontal mode MODE2, the gate driver 300 is located at the first location of the display panel 100 or the second location of the display panel 100.

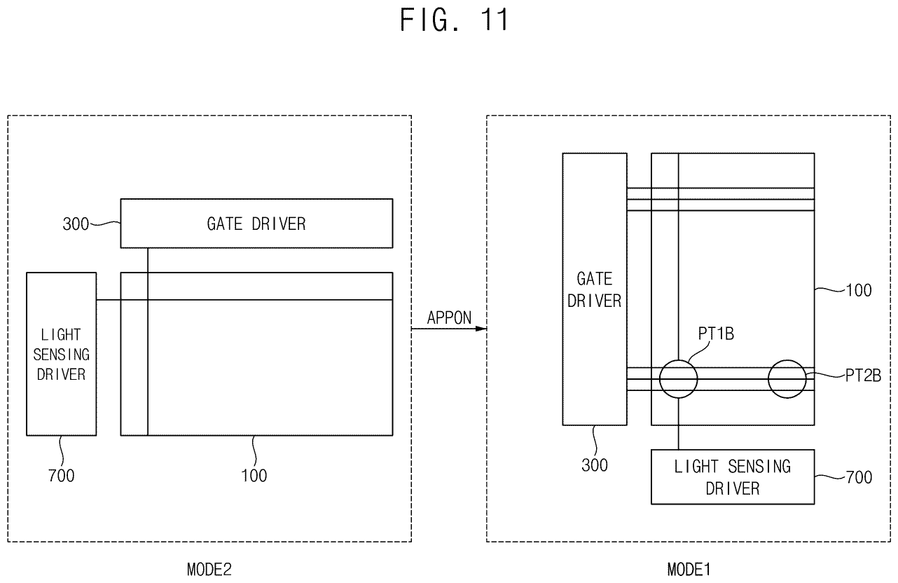

FIG. 11 illustrates mode conversion of a display apparatus of FIG. 1.

Referring to FIGS. 1, 8 and 11, in an embodiment, when the panel driver is driving the display panel 100 in the horizontal mode MODE2, the app-on signal APPON is applied.

In an embodiment, in response to the app-on signal APPON, the panel driver drives the display panel 100 in the vertical mode MODE1 and drives the display panel 100 such that it displays the guidance image CASE2. Accordingly, the display apparatus operates in the vertical mode MODE1 and displays the guidance image CASE2. Accordingly, the first sensing region PT1B and the second sensing region PT2B are displayed on the adjacent gate line group, so that the timing of gate signals applied to the first sensing region PT1B and the second sensing region PT2B is substantially the same. Accordingly, an influence of different gate signal timings is reduced. Accordingly, the accuracy of the sensing current is further increased.

The accuracy of the sensing current is increased, which increases an accuracy of the PPG signals PPGD. Accordingly, an accuracy of the biomarker is increased.

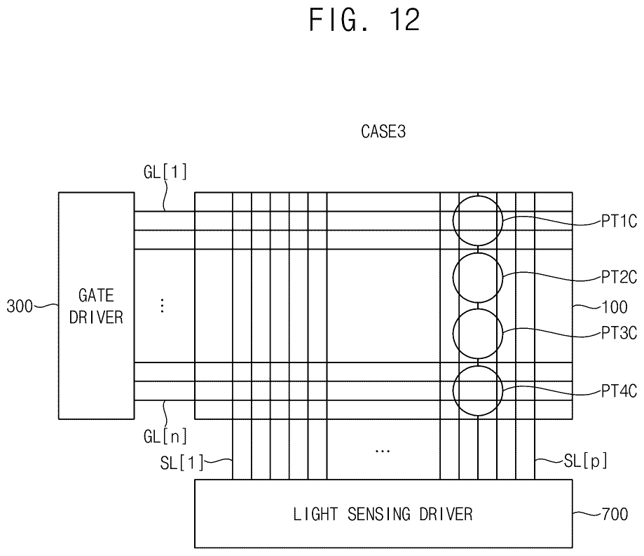

FIG. 12 illustrates an example of a guidance image CASE3 output by a display apparatus of FIG. 1.

Referring to FIG. 12, a guidance image CASE3 is substantially the same as the guidance image CASE1 of FIG. 5 except that the guidance image CASE3 further includes a third sensing region PT3C and a fourth sensing region PT4C, so that the same reference numerals may be used to refer to the same elements and a repetitive description concerning the above elements may be omitted.

In an embodiment, some pixels of the adjacent vertical pixel group display the third sensing region PT3C and the fourth sensing region PT4C. The third PPG signals that correspond to a third finger of the user are generated by the third sensing region PT3C. The fourth PPG signals that correspond to a fourth finger of the user are generated by the fourth sensing region PT4C. A first sensing region PT1C, a second sensing region PT2C, the third sensing region PT3C and the fourth sensing region PT4C are displayed on the adjacent sensing line group. Accordingly, adjacently located sensing lines SL output the sensing current. Accordingly, an accuracy of the sensing current is increased, which increases an accuracy of the sensing current, which increases an accuracy of the PPG signals PPGD. In addition, the number of the PPG signals that correspond to the fingers, such as the first to fourth fingers, is increased, which further increases an accuracy of the biomarker.

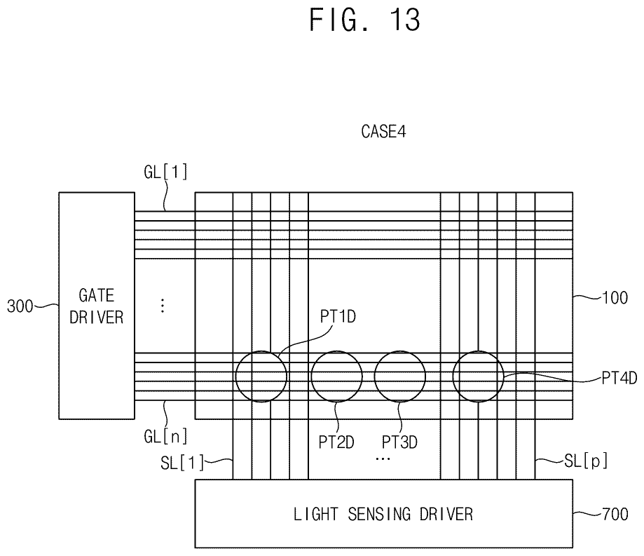

FIG. 13 illustrates an example of a guidance image CASE4 output by a display apparatus of FIG. 1.

Referring to FIG. 13, in an embodiment, a guidance image CASE4 is substantially same as the guidance image CASE2 of FIG. 7 except that the guidance image CASE4 further includes a third sensing region PT3D and a fourth sensing region PT4D, so that the same reference numerals may be used to refer to the same elements and a repetitive description concerning the above elements may be omitted.

In an embodiment, some pixels of the adjacent horizontal pixel group display the third sensing region PT3D and the fourth sensing region PT4D. The third PPG signals that correspond to the user's third finger are generated by the third sensing region PT3D. The fourth PPG signals that correspond to the user's fourth finger are generated by the fourth sensing region PT4D. A first sensing region PT1D, a second sensing region PT2D, the third sensing region PT3D and the fourth sensing region PT4D are displayed on the adjacent gate line group. In an embodiment, the gate signal applied to the gate lines is sequentially output to the first to N-th gate lines. Accordingly, a timing of a gate signal applied to the first gate line GL[1] and a timing of a signal applied to the N-th gate line GL[n] differ.

In an embodiment, the first sensing region PT1D, the second sensing region PT2D, the third sensing region PT3D and the fourth sensing region PT4D are displayed on the adjacent gate line group, so that a timing of the gate signals applied to the first sensing region PT1D, the second sensing region PT2D, the third sensing region PT3D and the fourth sensing region PT4D is substantially the same. Accordingly, an influence of deviations due to different gate signal timings is reduced, which increases an accuracy of the sensing current.

Accordingly, an accuracy of the sensing current is increased. The accuracy of the sensing current increased, which increases an accuracy of the PPG signals PPGD. In addition, the number of the PPG signals that correspond to the fingers, such as the first to fourth fingers, is increased, which further increases an accuracy of the biomarker.

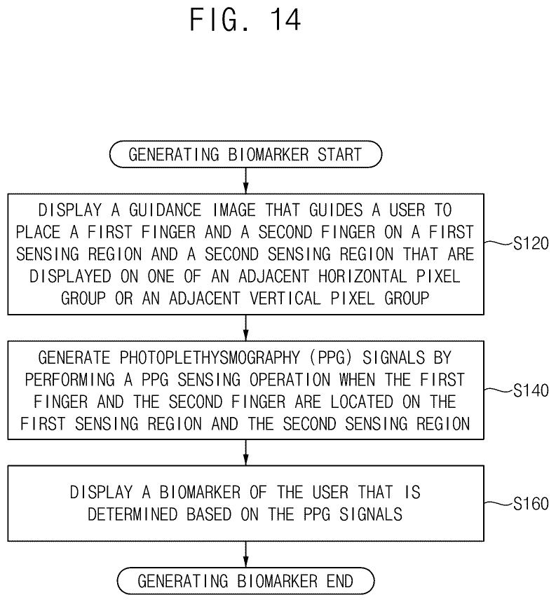

FIG. 14 is a flowchart of a method of generating a biomarker.

Referring to FIG. 14, the display apparatus that generates the biomarker displays a guidance image that guides a user to respectively place a first finger and a second finger on a first sensing region and a second sensing region that are displayed on one of an adjacent horizontal pixel group or an adjacent vertical pixel group (S120). The display apparatus generates the PPG signals by performing a PPG sensing operation when the first finger and the second finger are respectively located on the first sensing region and the second sensing region (S140). The display apparatus displays a biomarker of the user that is determined based on the PPG signals (S160).

In an embodiment, one of the adjacent horizontal pixel group or the adjacent vertical pixel group displays the first sensing region and the second sensing region. Accordingly, the accuracy of the sensing current is increased. The accuracy of the sensing current is increased, which increases the accuracy of the PPG signals. Accordingly, the accuracy of the biomarker is increased.

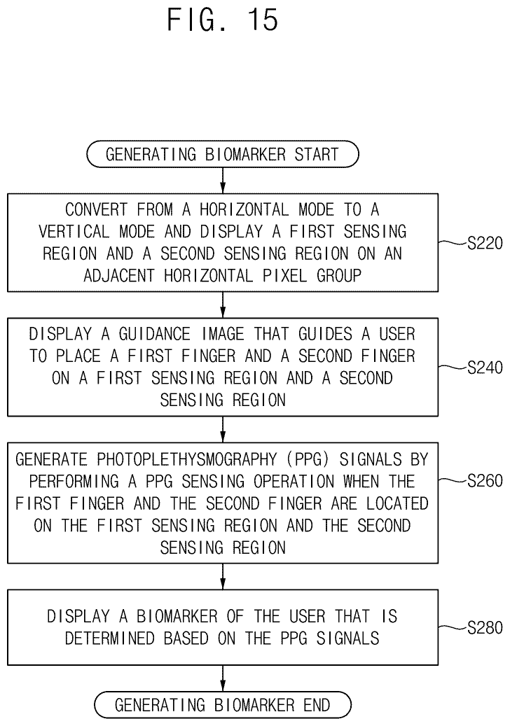

FIG. 15 is a flowchart a method of generating a biomarker.

Referring to FIG. 15, in an embodiment, when the display apparatus that generates the biomarker is driven in the horizontal mode the display apparatus converts to the vertical mode and displays the first sensing region and the second sensing region on an adjacent horizontal pixel group (S220). The display apparatus displays a guidance image that guides a user to respectively place a first finger and a second finger on a first sensing region and a second sensing region that are displayed on one of an adjacent horizontal pixel group or an adjacent vertical pixel group (S240). The display apparatus generates the PPG signals by performing the PPG sensing operation when the first finger and the second finger are respectively located on the first sensing region and the second sensing region (S260). The display apparatus displays a biomarker of the user that is determined based on the PPG signals (S280). In an embodiment, a process of converting to the vertical mode and displaying the first sensing region and the second sensing region on an adjacent vertical pixel group (S220) and a process of displaying a guidance image that guides a user to respectively place a first finger and a second finger on a first sensing region and a second sensing region that are displayed on one of an adjacent horizontal pixel group or an adjacent vertical pixel group (S240) may be simultaneously performed.

In an embodiment, the adjacent vertical pixel group displays the first sensing region and the second sensing region. Accordingly, the first sensing region and the second sensing region are displayed on the adjacent gate line group, so that the timing of the gate signals applied to the first sensing region and the second sensing region is substantially the same. Accordingly, an influence of deviations due to different gate signal timings is reduced, which increases an accuracy of the sensing current.

Accordingly, an accuracy of the sensing current is increased. The accuracy of the sensing current is increased, which increases an accuracy of the PPG signals. In addition, the accuracy of the biomarker is increased.

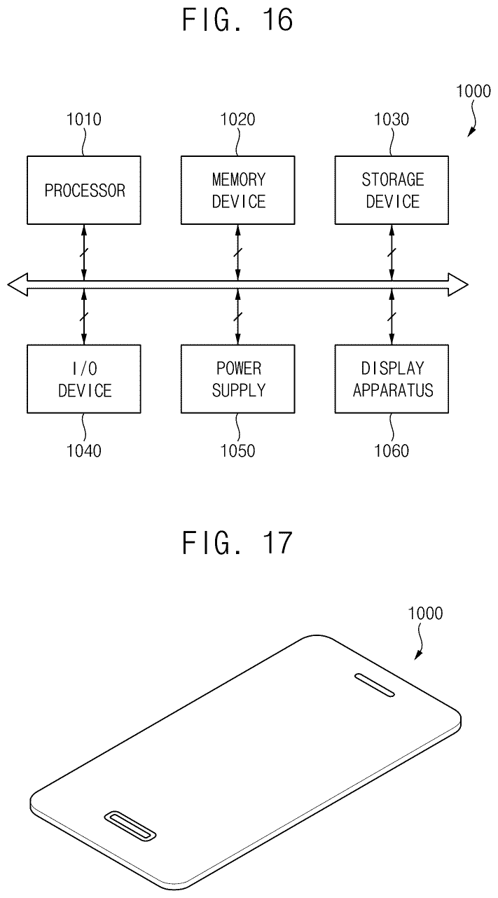

FIG. 16 is a block diagram of an electronic apparatus according to an embodiment of the present inventive concept. FIG. 17 illustrates an example in which the electronic apparatus of FIG. 16 is implemented as a smart phone.

Referring to FIGS. 16 and 17, in an embodiment, the electronic apparatus 1000 includes a processor 1010, a memory device 1020, a storage device 1030, an input/output (I/O) device 1040, a power supply 1050, and a display apparatus 1060. The display apparatus 1060 is the display apparatus of FIG. 1. In addition, the electronic apparatus 1000 further includes a plurality of ports for communicating with a video card, a sound card, a memory card, a universal serial bus (USB) device, and other electronic apparatuses, etc.

In an embodiment, as illustrated in FIG. 17, the electronic apparatus 1000 can be implemented as a smart phone. However, the electronic apparatus 1000 is not necessarily limited thereto. For example, the electronic apparatus 1000 may be implemented as a cellular phone, a video phone, a smart pad, a smart watch, a tablet PC, a car navigation system, a computer monitor, a laptop, a head mounted display (HMD) device, etc.

The processor 1010 performs various computing functions or tasks. The processor 1010 may be one of a micro-processor, a central processing unit (CPU), an application processor (AP), etc. The processor 1010 is coupled to other components by at least one of an address bus, a control bus, a data bus, etc. Further, the processor 1010 may be coupled to an extended bus such as a peripheral component interconnection (PCI) bus.

The processor 1010 outputs the input image data IMG, the app-on signal APPON and the input control signal CONT to the driving controller 200 of FIG. 1.

The memory device 1020 stores data for operations of the electronic apparatus 1000. For example, the memory device 1020 includes at least one non-volatile memory device such as an erasable programmable read-only memory (EPROM) device, an electrically erasable programmable read-only memory (EEPROM) device, a flash memory device, a phase change random access memory (PRAM) device, a resistance random access memory (RRAM) device, a nano floating gate memory (NFGM) device, a polymer random access memory (PoRAM) device, a magnetic random access memory (MRAM) device, a ferroelectric random access memory (FRAM) device, etc., and/or at least one volatile memory device such as a dynamic random access memory (DRAM) device, a static random access memory (SRAM) device, a mobile DRAM device, etc.

The storage device 1030 includes one or more of a solid state drive (SSD) device, a hard disk drive (HDD) device, a CD-ROM device, etc. The I/O device 1040 includes an input device such as a keyboard, a keypad, a mouse device, a touch-pad, a touch-screen, etc., and an output device such as a printer, a speaker, etc. In some embodiments, the display apparatus 1060 is included in the I/O device 1040. The power supply 1050 provides power for operations of the electronic apparatus 1000. The display apparatus 1060 is coupled to other components by the buses or other communication links.

The display apparatus according to embodiments can be incorporated into a computer, a notebook, a mobile phone, a smart phone, a smart pad, a PMP, a PDA, an MP3 player, etc.

The foregoing is illustrative of embodiments of the present inventive concept and is not to be construed as necessarily limiting thereof. Although a few embodiments of the present inventive concept have been described, those skilled in the art will readily appreciate that many modifications are possible in the embodiments without materially departing from the novel teachings and advantages of the present inventive concept. Accordingly, all such modifications are intended to be included within the scope of the present inventive concept as defined in the claims. Therefore, it is to be understood that the foregoing is illustrative of the present inventive concept and is not to be construed as limited to the specific embodiments disclosed, and that modifications to the disclosed embodiments, as well as other embodiments, are intended to be included within the scope of the appended claims. The present inventive concept is defined by the following claims, with equivalents of the claims to be included therein.

What is claimed is:

1. A display apparatus, comprising:
a display panel that includes a plurality of pixels; and
a panel driver that drives the display panel,
wherein the pixels include an adjacent horizontal pixel group that includes first to second pixel-rows and an adjacent vertical pixel group that includes first to second pixel-columns,
wherein the pixels include an emitting pixel that includes a light emitting element and a light sensing pixel that includes a photodiode,
wherein the panel driver drives the display panel such that the display panel displays a first guidance image that guides a user to place fingers on a first sensing region and a second sensing region of the display panel,
wherein the light sensing pixel outputs a sensing current that generates photoplethysmography (PPG) signals by sensing the user's fingers, and
wherein the first sensing region and the second sensing region are displayed on one of the adjacent horizontal pixel group or the adjacent vertical pixel group.

2. The display apparatus of claim 1, wherein the first sensing region and the second sensing region are displayed on the adjacent vertical pixel group.

3. The display apparatus of claim 2, wherein the panel driver includes:
a gate driver that applies a gate signal to the display panel through a plurality of gate lines; and
a light sensing driver that receives the sensing current from the pixels through a plurality of sensing lines,
wherein the gate lines include an adjacent gate line group that includes first to second gate lines,
wherein the sensing lines include an adjacent sensing line group that includes first to second sensing lines, and
wherein the adjacent vertical pixel group is connected to the adjacent sensing line group.

4. The display apparatus of claim 3, wherein the light sensing driver receives the sensing current from the adjacent sensing line group connected to the adjacent vertical pixel group.

5. The display apparatus of claim 3,
wherein the sensing lines include first to P-th sensing lines, where Pis a positive integer,
wherein the first to P-th sensing lines are sequentially disposed on the display panel, and
wherein the adjacent sensing line group includes at least two adjacent sensing lines of the first to P-th sensing lines.

6. The display apparatus of claim 3, wherein the gate lines extend in a first direction and the sensing lines extend in a second direction different from the first direction.

7. The display apparatus of claim 2,
wherein the panel driver drives the display panel such that the display panel displays a second guidance image that guides the user to place fingers on the first sensing region, the second sensing region, a third sensing region and a fourth sensing region of the display panel, and
wherein the third sensing region and the fourth sensing region are displayed on the adjacent vertical pixel group.

8. The display apparatus of claim 1, wherein the first sensing region and the second sensing region are displayed on the adjacent horizontal pixel group.

9. The display apparatus of claim 8, wherein the panel driver includes:
a gate driver that applies a gate signal to the display panel through a plurality of gate lines; and
a light sensing driver that receives the sensing current from the pixels through a plurality of sensing lines,
wherein the gate lines include an adjacent gate line group that includes first to second gate lines,
wherein the sensing lines include an adjacent sensing line group that include first to second sensing lines, and
wherein the adjacent horizontal pixel group is connected to the adjacent gate line group.

10. The display apparatus of claim 9, wherein the light sensing driver receives the sensing current from the sensing lines connected to the adjacent horizontal pixel group.

11. The display apparatus of claim 9,
wherein the sensing lines include first to N-th gate lines, where N is a positive integer,
wherein the first to N-th gate lines are sequentially disposed on the display panel, and
wherein the adjacent gate line group includes at least two adjacent gate lines of the first to N-th gate lines.

12. The display apparatus of claim 9, wherein the gate lines extend in a first direction and the sensing lines extend in a second direction different from the first direction.

13. The display apparatus of claim 8,
wherein the panel driver drives the display panel such that the display panel displays a third guidance image that guides the user to place fingers on the first sensing region, the second sensing region, a third sensing region and a fourth sensing region of the display panel, and
wherein the third sensing region and the fourth sensing region are displayed on the adjacent horizontal pixel group.

14. The display apparatus of claim 8,
wherein the panel driver drives the display panel in a horizontal mode or a vertical mode, and
wherein when the display panel displays the guidance image in the horizontal mode, the panel driver converts the display panel from the horizontal mode to the vertical mode in response to receipt of an app-on signal.

15. A method of operating a display apparatus, the method comprising:
displaying a guidance image that guides a user to respectively place a first finger and a second finger on a first sensing region and a second sensing region that are displayed on one of an adjacent horizontal pixel group that includes first to second pixel-rows or an adjacent vertical pixel group that includes first to second pixel-columns;
generating photoplethysmography (PPG) signals by performing a PPG sensing operation when the first finger and the second finger are respectively located on the first sensing region and the second sensing region; and
displaying a biomarker of the user that is determined based on the PPG signals.

16. The method of claim 15, further comprising:
converting a mode of the display apparatus from a horizontal mode to a vertical mode in response to receipt of an app-on signal when the display apparatus is driven in the horizontal mode; and
displaying the first sensing region and the second sensing region on the adjacent horizontal pixel group in the vertical mode.

17. The method of claim 15, wherein the first sensing region and the second sensing region are displayed on the adjacent vertical pixel group.

18. The method of claim 17, wherein the adjacent vertical pixel group includes at least two adjacent pixel-columns of first to P-th pixel-columns included in a display panel, where P is a positive integer.

19. The method of claim 15, wherein the first sensing region and the second sensing region are displayed on the adjacent horizontal pixel group.

20. The method of claim 19, wherein the adjacent horizontal pixel group includes at least two adjacent pixel-rows of first to N-th pixel-rows, where Nis a positive integer.
